(19) United States
(12) Patent Application Publication
(10) Pub. No.: US 2022/0273631 A1
(43) Pub. Date: Sep. 1, 2022

(54) METHOD FOR TREATING SEXUAL DYSFUNCTION

(71) Applicant: Catalyst Pharmaceuticals, Inc., Coral Gables, FL (US)

(72) Inventors: Stanley IYADURAI, Coral Gables, FL (US); Steven Miller, Coral Gables, FL (US)

(73) Assignee: Catalyst Pharmaceuticals, Inc., Coral Gables, FL (US)

(21) Appl. No.: 17/633,021

(22) PCT Filed: Aug. 3, 2020
(86) PCT No.: PCT/US2020/044745

§ 371 Date: Feb. 4, 2022

Related U.S. Application Data
(60) Provisional application No. 62/883,295, filed on Aug. 6, 2019.

(51) Int. Cl. A61K 31/44; A61P 25/16; A61P 15/00

(52) U.S. Cl. CPC A61K 31/44; A61P 25/16; A61P 15/00

(57) ABSTRACT

The present disclosure relates to a method of treating female or male sexual dysfunction, wherein the method comprises orally administering to a female or male subject an effective amount of 3,4-diaminopyridine or a pharmaceutically acceptable salt thereof.

### BACKGROUND

Sexual dysfunction is a difficulty experienced by an individual (male or female) or a couple during any stage of normal sexual activity. Sexual dysfunction can affect, for example, desire, physical pleasure, arousal, and/or the ability to orgasm. According to Diagnostic and Statistical Manual of Mental Disorders, Fifth Edition (DSM-5), sexual dysfunction requires a person to feel extreme distress and interpersonal strain for a minimum of six months. The origin of the dysfunction may stem from a variety of conditions, such as from biological conditions, organic conditions, psychological conditions, and/or social conditions.

Female sexual dysfunction affects 41% of reproductive-age women worldwide, making it a highly prevalent medical issue (McCool-Myers, M., et al., BMC Women's Health 18(108):1-15 (2018)). A number of biological, psychological, and social factors play a role in the prevalence of female sexual dysfunction. Male sexual dysfunction has long been known to be common and knowledge of normal male sexual function and the causes of sexual dysfunction have become better understood. Impaired sexual function (e.g., problems with sexual desire, arousal, orgasm and sexual pain) causes high levels of personal or interpersonal distress.

3,4-Diaminopyridine, also known as amifampridine or 3,4-DAP, is a potassium channel blocker that has been used as a drug in the treatment of a number of rare muscle diseases, such as congenital myasthenic syndrome and Lambert-Eaton myasthenic syndrome (LEMS). 3,4-Diaminopyridine free base form is sold under the trade name Ruzurgi® which is approved to treat LEMS in pediatric patients. The phosphate salt of 3,4-diaminopyridine is sold under the trade name Firdapse® and is approved to treat LEMS in adults.

There is a need for a treatment for both female and male sexual dysfunction, that will enhance desire and sexual satisfaction, and improve the overall sexual life of the female or male subject in need of such treatment.

### BRIEF SUMMARY

The present disclosure relates to the use of 3,4-diaminopyridine and its pharmaceutically acceptable salts in the treatment of sexual dysfunction. Specifically, the present disclosure relates to a method of treating female sexual dysfunction or male sexual dysfunction, wherein the method comprises orally administering to a female or male subject in need thereof an effective amount of 3,4-diaminopyridine or a pharmaceutically acceptable salt thereof.

In one aspect, the present disclosure provides a method of treating sexual dysfunction in a subject in need thereof. The method comprises orally administering to the subject in need of the treatment an effective amount of 3,4-diaminopyridine or a pharmaceutically acceptable salt thereof. In some embodiments of this aspect, the method comprises orally administering to the subject in need of the treatment an effective amount of 3,4-diaminopyridine phosphate salt.

In some embodiments, the sexual dysfunction is a disorder selected from the group consisting of female sexual interest/arousal disorder, female orgasmic disorder, other female sexual dysfunction, male hypoactive sexual desire disorder, delayed ejaculation, erectile disorder, and other male sexual dysfunction.

In another aspect, the present disclosure provides a method of increasing sexual desire in a subject in need thereof, comprising orally administering to the subject an effective amount of 3,4-diaminopyridine or a pharmaceutically acceptable salt thereof. In some embodiments of this aspect, the method comprises orally administering to the subject in need of the treatment an effective amount of 3,4-diaminopyridine phosphate salt.

In another aspect, the present disclosure provides a method of increasing orgasmic ability in a subject in need thereof, comprising orally administering to the subject an effective amount of 3,4-diaminopyridine or a pharmaceutically acceptable salt thereof. In some embodiments of this aspect, 3,4-diaminopyridine phosphate salt is administered.

In another aspect, the present disclosure provides a method of increasing sexual satisfaction in a subject in need thereof, comprising orally administering to the subject an effective amount of 3,4-diaminopyridine or a pharmaceutically acceptable salt thereof. In some embodiments, 3,4-diaminopyridine phosphate salt is administered.

In some embodiments, the subject is a female subject. In some embodiments, the subject is a male subject.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### DETAILED DESCRIPTION

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### Definitions

For convenience, the meaning of some terms and phrases used in the specification, examples, and appended claims are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

The articles “a,” “an,” and “the” are used herein to refer to one or to more than one (i.e., to at least one) of the grammatical object of the article. By way of example, “an element” means one element or more than one element.

As used herein, the term “about” means ±10% of the specified value, unless otherwise indicated.

The term “at least” prior to a number or series of numbers is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, the terms “comprises,” “comprising,” “having,” “including,” “containing,” and the like are open-ended terms meaning “including, but not limited to.” To the extent a given embodiment disclosed herein “comprises” certain elements, it should be understood that present disclosure also specifically contemplates and discloses embodiments that “consist essentially of” those elements and that “consist of” those elements.

As used herein the terms “consists essentially of,” “consisting essentially of,” and the like are to be construed as a semi-closed terms, meaning that no other ingredients which materially affect the basic and novel characteristics of an embodiment are included.

As used herein, the terms “consists of,” “consisting of,” and the like are to be construed as closed terms, such that an embodiment “consisting of” a particular set of elements excludes any element, step, or ingredient not specified in the embodiment.

The terms “treat,” “treating,” and “treatment” refer to any indicia of success in the treatment or amelioration of an injury, disease, or condition, including any objective or subjective parameter such as abatement; remission; diminishing of symptoms or making the injury, disease, or condition more tolerable to the patient; slowing in the rate of degeneration or decline; or improving a patient's physical or mental well-being. The treatment or amelioration of symptoms can be based on objective or subject parameters, including the results of a physical examination, neuropsychiatric examinations, or psychiatric evaluation.

By an “effective” amount or a “therapeutically effective amount” of a drug or pharmacologically active agent is meant a nontoxic but sufficient amount of the drug or agent to provide the desired effect. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The term “pharmaceutically acceptable salt” refers to salts prepared from pharmaceutically acceptable inorganic and organic acids.

The term “sexual dysfunction” as used herein refers to any female or male sexual dysfunction including, without limitation, impairment in sexual desire, arousal, orgasm, or satisfaction, or premature ejaculation, any or all of which may be due to, for example, psychogenic, biologic (including vasogenic, endocrine related, menopause, and neurologic disorders), or medication-induced mechanisms, excluding pain or sexual paraphilias. The sexual dysfunction can be, for example, a disorder selected from the group consisting of a female sexual interest/arousal disorder, a female orgasmic disorder, other female sexual dysfunction, a male hypoactive sexual desire disorder, delayed ejaculation, erectile disorder, and other male sexual dysfunction.

The term “female sexual interest/arousal disorder” or “FSIAD” refers to a disorder marked by lack of, or significantly reduced, sexual interest/arousal. A woman must have three of the following six symptoms in order to receive the diagnosis: absent or reduced interest in sexual activity; absent or reduced sexual thoughts or fantasies; no or reduced initiation of sexual activity, and typically unreceptive to a partner's attempts to initiate; absent or reduced sexual excitement or pleasure in almost all or all sexual encounters; absent or reduced sexual interest/arousal in response to any internal or external sexual cues; and absent or reduced genital or non-genital sensations during sexual activity in all or almost all sexual encounters. These symptoms must cause clinically significant distress and have persisted for a minimum of six months. The disorder is specified by severity level and subtyped into lifelong versus acquired, generalized versus situational. FSIAD is a combination of Female Hypoactive Desire Disorder and Female Arousal Disorder from the DSM-IV.

The term “female orgasmic disorder” refers to a disorder marked by a significant change in orgasm such as reduced intensity, delay, infrequency, or absence of orgasm. The symptom must last for at least six months and not be related to other physical, mental or relational problem. Although no one cause has been identified, female orgasmic disorder has been associated with relational problems, stress, depression, anxiety, medications and chronic medical conditions.

The term “orgasm”, also known as “sexual climax”, refers to a sudden discharge of accumulated sexual excitement during the sexual response cycle, resulting in rhythmic muscular contractions in the pelvic region characterized by sexual pleasure. Orgasms are experienced by males and females and they are controlled by the involuntary or autonomic nervous system. They are often associated with other involuntary actions, including muscular spasms in multiple areas of the body, a general euphoric sensation and, frequently, body movements and vocalizations.

The term “orgasmic ability” refers to a subject's tendency to experience orgasms.

The term “increasing orgasmic ability” refers to increasing a subject's tendency to experience orgasms.

The term “sexual satisfaction” is an important indicator of sexual health and is strongly associated with relationship satisfaction. Sexual satisfaction is positively associated with indicators of relationship quality such as love, commitment, and stability. For example, female sexual satisfaction can be measured by the Sexual Satisfaction Scale for Women (SSS-W) which is a 30-item self-report inventory designed to measure sexual satisfaction and sexual distress composed of 5 domains supported by factor analyses: contentment, communication, compatibility, relationship concern, and personal concern. See, e.g., Meston, et al., J. Sex Med. 2(1):66-81 (2005).

The term “other female sexual dysfunction” refers to substance/medication-induced sexual dysfunction, or unspecified sexual dysfunction.

The term “substance/medication-induced sexual dysfunction” refers to alcohol- or drug-induced sexual problems.

The term “unspecified sexual dysfunction” refers to a sexual dysfunction where the subject exhibits symptoms from several sexual disorders at the same time. Symptoms include some or all of those found in other sexual dysfunctions.

The term “primary sexual dysfunction” refers to a sexual dysfunction that is a direct result of neurologic changes that affect the sexual response.

The term “secondary sexual dysfunction” refers to a sexual dysfunction that stems from symptoms that do not directly involve nervous pathways to the genital system, such as bladder and bowel problems, fatigue, spasticity, muscle weakness, body or hand tremors, impairments in attention and concentration, and non-genital sensory changes.

The term “male hypoactive sexual desire disorder” or “MHSDD” refers to a disorder marked by persistent or recurrently deficient sexual or erotic thoughts, fantasies, and desire for sexual activity. These symptoms must have persisted for a minimum of six months, and they must cause clinically significant distress.

The term “delayed ejaculation” or “DE” refers to a persistent difficulty or inability to achieve orgasm despite the presence of adequate desire, arousal, and stimulation. In order to be diagnosed with the disorder, patients must present with one of two symptoms: either a delay in or an infrequency of ejaculation on 75-100% of occasions for at least six months. The disorder can be specified as lifelong or acquired as well as generalized or situational. Most commonly, the term refers to a condition in which a man is unable to orgasm with his partner, even though he is able to achieve and maintain an erection. Typically, men who present with DE are able to ejaculate during masturbation or sleep.

The term “erectile disorder” is also known as “erectile dysfunction”, “impotence”, or “ED”, refers to the inability to get or maintain an erection that is firm enough for sexual intercourse or other satisfying sexual activity. While it is normal to occasionally lose an erection, men with ED have a chronic problem. A diagnosis of male erectile disorder will manifest at least one of three symptoms 75% to 100% of the time during sexual activity: 1) men will struggle to achieve an erection during sexual activity; or 2) men will struggle maintaining an erection until the completion of sexual activity; or 3) there will be a noticeable decrease in erectile rigidity. The diagnosis requires persistence of these symptoms for approximately six months. Inability to get an erection during sexual activity. A five-part questionnaire, known as the International Index of Erectile Function (IIEF-5), rates symptoms and helps determine the severity of dysfunction. Symptoms can be situational, which means they occur only in specific situations or with specific partners. Symptoms can also be generalized, meaning they occur all the time, regardless of the situation or partner involved. Low self-esteem, lack of confidence, and fear of sexual relations often accompany the experience of erectile dysfunction. The ED can be a primary ED or a secondary ED.

The term “primary ED” or “primary erectile disorder” refers to a disorder that occurs when a man has never been able to achieve an erection.

The term “secondary ED” or “secondary erectile disorder” refers to a disorder which develops later in life after a long period of being able to maintain an erection. Some of the most common causes of secondary ED include: psychological distress, such as body image problems or anxiety about sex; relationship problems; blood vessel disorders, including those due to heart health issues; neurological damage, which is often due to diabetes; prostate disorders; and smoking-induced atherosclerosis, which clogs arteries. Periodic difficulties with getting or sustaining an erection are typical and often due to stress.

A man who only experiences occasional ED is unlikely to be diagnosed with ED or a related medical condition. A number of health problems can cause erectile dysfunction by damaging the nerves or narrowing the blood vessels. When blood vessels narrow, it becomes harder for the penis to fill with blood. Some common causes include: cardiovascular disease, atherosclerosis, high blood pressure, diabetes, spinal cord injuries, nerve damage due to surgery, and prostatitis and other prostate disorders. Some mental health conditions, including anxiety disorders and depression, may also play a role. Some men find that certain drugs, including antidepressants and blood pressure medications, make it more difficult to achieve or sustain an erection.

The term “autonomic dysfunction”, also known as “dysautonomia” or “autonomic neuropathy,” refers to a condition in which the autonomic nervous system (ANS) does not work properly. This may affect the functioning of the heart, bladder, intestines, sweat glands, pupils, smooth muscles, and blood vessels. Dysautonomia has many causes, not all of which may be classified as neuropathic.

The term “other male sexual dysfunction” refers to substance/medication-induced sexual dysfunction, or unspecified sexual dysfunction.

The term “anorgasmia” refers to a sexual dysfunction in which a person cannot achieve orgasm despite adequate stimulation.

The term “primary anorgasmia” refers to a condition where a person has never experienced an orgasm.

The term “secondary anorgasmia” refers to the loss of the ability to have orgasms or loss of the ability to reach orgasm of past intensity.

The term “situational anorgasmia” refers to a condition where a person is orgasmic in some situations but may not be in others. For example, the person may have an orgasm from one type of stimulation but not from another or achieve orgasm with one partner but not another.

The term “pre-menopausal” refers to a woman who has no symptoms of going through perimenopause or menopause. The pre-menopausal woman can still have periods (whether they're regular or irregular) and is considered to be in her reproductive years. Some hormonal changes may be occurring, but there are no noticeable changes in the body.

The term “perimenopause” refers to a period where a woman will start to experience symptoms of menopause (for example, changes in period cycle, hot flashes, sleep disturbances, or mood swings). Perimenopause can begin eight to 10 years before menopause, when the ovaries gradually produce less estrogen. It usually starts in a woman's 40s, but can start in the 30s as well. Perimenopause lasts up until menopause, the point when the ovaries stop releasing eggs. In the last one to two years of perimenopause, the drop in estrogen accelerates. At this stage, many women can experience menopause symptoms. Perimenopausal women are still having menstrual cycles during this time, and can get pregnant.

The term “menopausal” refers to a woman who is at the point where she no longer has menstrual periods. At this stage, the ovaries have stopped releasing eggs and producing most of their estrogen. Menopause is diagnosed when a woman has gone without a period for 12 consecutive months.

The term “post-menopausal” refers to a woman after menopause, i.e., after the period of time the woman has experienced 12 consecutive months without menstruation. At post-menopause, menopausal symptoms, such as hot flashes, can ease for many women.

The term “diabetic neuropathy” refers to a serious and common complication of type 1 and type 2 diabetes. It's a type of nerve damage caused by long-term high blood sugar levels.

The “Female Sexual Functioning Index” or “FSFI” is a 19-item self-report inventory designed to assess female sexual function. It comprises six domains: desire (two items)], arousal (four items), lubrication (four items), orgasm, satisfaction, and pain (three items each).

The “Brief Male Sexual Function Inventory” or “BSFI” is a self-report inventory designed to help evaluate male sexual dysfunction in the light of symptoms pertaining to conditions of the lower urinary tract. The original study recommends to the patient to think of his experiences for the past month and is preceded by this indication: “Lets define sexual drive as a feeling that may include wanting to have a sexual experience (masturbation or intercourse) thinking about having sex or felling frustrated due to lack of sex.” The BSFI consists of 11 items divided in 5 functional domains, as described in Table 1 below:

TABLE 1

| BSFI functional domain | BSFI items description |
|---|---|
| Sexual drive | 1. Frequency of experiencing sexual drive; |
| | 2. Rating the level of sexual drive; |
| Erections score | 3. Frequency of partial or full sexual |
| | erections during stimulation; |
| | 4. Frequency of the ability to have sexual |
| | intercourse (firm erections); |
| | 5. Difficulty in obtaining an erection; |
| Ejaculation score | 6. Degree of difficulty ejaculating while |
| | being sexually stimulated; |
| | 7. Considering the amount of semen |
| | ejaculated; |
| Problem assessment | 8. To what extent is lack of sex drive |
| score | considered to be a problem; |
| | 9. To what extent is the ability to keep an |
| | erection a problem; |
| | 10. To what extent ejaculation is considered a |
| | problem; |
| Overall satisfaction | 11. Overall satisfaction with sexual life for |
| score | past 30 days. |

The “International Index of Erectile Function” or “IIEF” is a self-report inventory consisting of 15 questions and 5 functional domains, which was developed for use in determining efficacy of treatment in controlled clinical trials. The IIEF has high sensitivity and specificity for detecting real treatment effects or the lack of treatment effects in patients with ED of broad-spectrum etiology. A score of 0-5 is awarded to each of the 15 questions that examine the 4 main domains of male sexual function: erectile function, orgasmic function, sexual desire, and intercourse satisfaction. Rosen, R., et al. (in Urology 49:822-830 (1997)) describe a study where the IIEF was tested in a series of 111 men with sexual function and 109 age-matched, normal volunteers. The following mean scores presented in Table 2 were recorded.

TABLE 2

| | Maximum | | |
|---|---|---|---|
| Function Domain | Score | Controls | Patients |
| A. Erectile Function (questions 1-5 and | 30 | 25.8 | 10.7 |
| 15) | | | |
| B. Orgasmic Function (questions 9 and | 10 | 9.8 | 5.3 |
| 10) | | | |
| C. Sexual Desire (questions 11 and 12) | 10 | 7.0 | 6.3 |
| D. Intercourse Satisfaction (questions | 15 | 10.6 | 5.5 |
| 6-8) | | | |
| E. Overall Satisfaction (questions 13 | 10 | 8.6 | 4.4 |
| and 14) | | | |

The term “micronization” as used herein refers to the process of reducing the average diameter of a solid material's particles. Traditional techniques for micronization focus on mechanical means, such as milling and grinding. Modern techniques make use of the properties of supercritical fluids and manipulate the principles of solubility. The term micronization usually refers to the reduction of average particle diameters to the micrometer range, but can also describe further reduction to the nanometer scale.

### Methods of the Disclosure

Impaired sexual function (e.g., problems with sexual desire, arousal, orgasm and sexual pain) can have a serious impact on the quality of life of the individual (male or female) or the couple afflicted with the condition.

In one aspect, the present disclosure provides a method of treating sexual dysfunction in a subject in need thereof, comprising orally administering to the subject an effective amount of 3,4-diaminopyridine or a pharmaceutically acceptable salt thereof. The sexual dysfunction experienced by the subject can be any female or male sexual dysfunction including, without limitation, impairment in: sexual desire, arousal, orgasm, or satisfaction, or premature ejaculation, any or all of which may be due to, for example, psychogenic, biologic (including vasogenic, endocrine related, menopause, and neurologic disorders), or medication-induced mechanisms, excluding pain or sexual paraphilias.

In some aspects, the sexual dysfunction is a disorder selected from the group consisting of a female sexual interest/arousal disorder, a female orgasmic disorder, other female sexual dysfunction, a male hypoactive sexual desire disorder, delayed ejaculation, erectile disorder, and other male sexual dysfunction.

In some aspects, the subject is a female subject. In some embodiments, the subject is an adult female subject.

In some embodiments, the sexual dysfunction is a female sexual interest/arousal disorder.

In some embodiments, the sexual dysfunction is a female orgasmic disorder. In some embodiments, the female orgasmic disorder is anorgasmia. In some embodiments, the anorgasmia is situational anorgasmia, primary anorgasmia, or secondary anorgasmia.

In some embodiments, the sexual dysfunction is other female sexual dysfunction.

In some embodiments, the other female sexual dysfunction is substance/medication-induced sexual dysfunction. In some embodiments, the other female sexual dysfunction is unspecified sexual dysfunction.

In some aspects, the subject is a male subject. In some embodiment, the subject is an adult male subject.

In some embodiments, the sexual dysfunction is a male hypoactive sexual desire disorder.

In some embodiments, the sexual dysfunction is delayed ejaculation.

In some embodiments, the sexual dysfunction is erectile disorder (ED).

The male sexual dysfunction can be a primary sexual dysfunction or a secondary sexual dysfunction. In some embodiments, the male sexual dysfunction is a primary sexual dysfunction. In some embodiments, the male sexual dysfunction is a secondary sexual dysfunction.

In certain embodiments, the secondary sexual dysfunction is secondary erectile disorder. In some embodiments, the secondary sexual dysfunction, such as ED, is caused by an autonomic dysfunction, a cardiac-related condition, or a blood-flow affecting medication. In some embodiments, the sexual dysfunction is caused by an autonomic dysfunction. An autonomic dysfunction can be caused by, for example, diabetes, multiple sclerosis, Parkinson's disease, brain or spinal tumor, stroke, temporal lobe epilepsy, an autoimmune disease, or alcoholism. In some embodiments, the autonomic dysfunction is diabetic autonomic neuropathy.

In some embodiments, the sexual dysfunction is other male sexual dysfunction.

In some embodiments, the other male sexual dysfunction is a substance/medication-induced sexual dysfunction. In other embodiments, the other male sexual dysfunction is an unspecified sexual dysfunction.

It has been surprisingly discovered that subjects suffering from sexual dysfunction have experienced overall improvement in their sexual functioning after daily oral administration of 3,4-diaminopyridine phosphate salt (3,4-DAPP). For example, in some embodiments, subjects provided with a Female Sexual Functioning Index (FSFI) before and after a four week course of an effective daily dose of 3,4-DAPP report increased sexual desire, increased orgasmic ability, and increased sexual satisfaction. Effective daily doses are discussed elsewhere herein.

In one aspect, the present disclosure provides a method of increasing sexual desire in a subject in need thereof, comprising orally administering to the subject an effective amount of 3,4-diaminopyridine or a pharmaceutically acceptable salt thereof. In certain embodiments of this aspect, 3,4-diaminopyridine free base is administered. In other embodiments, 3,4-diaminopyridine phosphate salt is administered. In still other embodiments, the hydrochloride salt of 3,4-diaminopyridine can be used.

In another aspect, the present disclosure provides a method of increasing orgasmic ability in a subject in need thereof, comprising orally administering to the subject an effective amount of 3,4-diaminopyridine or a pharmaceutically acceptable salt thereof. In certain embodiments of this aspect, 3,4-diaminopyridine free base is administered. In other embodiments, 3,4-diaminopyridine phosphate salt is administered. In still other embodiments, the hydrochloride salt of 3,4-diaminopyridine can be used.

In another aspect, the present disclosure provides a method of increasing sexual satisfaction in a subject in need thereof, comprising orally administering to the subject an effective amount of 3,4-diaminopyridine or a pharmaceutically acceptable salt thereof. Female sexual satisfaction can be measured, for example, by the SSS-W or the FSFI described above. In certain embodiments of this aspect, 3,4-diaminopyridine free base is administered. In other embodiments, 3,4-diaminopyridine phosphate salt is administered. In still other embodiments, the hydrochloride salt of 3,4-diaminopyridine can be used.

The therapeutic efficacy of 3,4-diaminopyridine and its salts in the present methods can be determined by clinicians, for example, by using appropriate self-report inventories or questionnaires. In some embodiments, the therapeutic efficacy of 3,4-diaminopyridine, or a pharmaceutically acceptable thereof, in the present methods in female subjects can be assessed by the Female Sexual Functioning Index (FSFI) designed to measure sexual functioning in women. See, e.g., Rosen et al., Journal of Sex and Marital Therapy 26:191-208 (2000).

In some embodiments, the therapeutic efficacy of 3,4-diaminopyridine, or a pharmaceutically acceptable thereof, in the present methods in male subjects can be assessed by the Brief Male Sexual Function Inventory (BSFI) described by, for example, Mykletun et al., BJU International 97:316-323 (2005). In some embodiments, the Male Sexual Health Questionnaire (MSHQ) can be used to assess sexual function and satisfaction in older men with urogenital symptoms of lower urinary tract and sexual dysfunction. See, e.g., Rosen, et al., Urology 64(4):777-782 (2004). In some embodiments, the therapeutic efficacy of 3,4-diaminopyridine, or a pharmaceutically acceptable thereof, in the present methods in male subjects, can be assessed by the 15-question International Index of Erectile Function (IIEF) described by, for example, Rosen et al., Urology 49:822-830 (1997).

In certain aspects, the subject is a female subject. In some embodiments, the subject is an adult female subject. In some embodiments, the female subject is a pre-menopausal, perimenopausal, menopausal, or post-menopausal woman.

In certain embodiments, the female subject is a pre-menopausal woman.

In certain embodiments, the female subject is a perimenopausal woman.

In certain embodiments, the female subject is a menopausal woman.

In certain embodiments, the female subject is a post-menopausal woman.

In certain aspects, the subject is a male subject. In some embodiments, the subject is an adult male subject.

In some embodiments, 3,4-diaminopyridine is administered as a phosphate salt.

In some embodiments, 3,4-diaminopyridine is administered as a tartrate salt. In some embodiments, 3,4-diaminopyridine is administered as a free base.

### Pharmaceutical Formulations

In some embodiments, the effective amount of 3,4-diaminopyridine or a pharmaceutically acceptable salt thereof is provided and/or administered in a pharmaceutical formulation suitable for oral administration.

In some embodiments, the oral formulation is selected from the group consisting of a solid, a semi-solid, and a liquid oral formulation. In some embodiments, the solid oral formulation is a tablet, a pill, a dragee, a powder, a granule, or a capsule. Suitable liquid formulations include, for example, aqueous suspensions, solutions, elixirs, and syrups. Suitable semi-solid formulations include, for example, oral gels.

Orally administered pharmaceutical formulations may contain conventional excipients known in the art and may be prepared by conventional methods. Orally administered pharmaceutical formulations of the disclosure can contain one or more pharmaceutically acceptable excipients. Suitable excipients include fillers such as saccharides, for example lactose or sucrose, mannitol, sodium saccharin or sorbitol, magnesiun carbonate, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents can be added such as the above-mentioned starches and also carboxymethyl-starch, cross-linked polyvinyl pyrrolidone, agar, or alginic acid or a salt thereof, such as sodium alginate and sodium starch glycolate. Suitable excipients also include flow-regulating agents and lubricants, for example, silica, talc, stearic acid or salts thereof, such as magnesium stearate or calcium stearate, and/or polyethylene glycol; sweetening agents such as fructose, aspartame or saccharin; flavoring agents such as peppermint, oil of wintergreen, or cherry; coloring agents; and preserving agents, to provide a pharmaceutically palatable preparation. In addition, dye stuffs or pigments can be added to the tablets or dragee coatings, for example, for identification or in order to characterize combinations of active compound doses. Other examples of suitable pharmaceutical excipients are described in Remington's Pharmaceutical Sciences pp. 1447-1676 (Alfonso R. Gennaro ed., 19th ed. 1995), incorporated herein by reference. In one embodiment, the excipients are of pharmaceutical grade.

In some embodiments, 3,4-diaminopyridine or a salt thereof can be micronized before preparing the oral formulations. Methods known in the art can be used for micronization of 3,4-diaminopyridine or its salts. For example, traditional micronization techniques based on friction to reduce particle size can be used, such as milling, bashing and grinding. A typical industrial mill is composed of a cylindrical metallic drum that usually contains steel spheres. As the drum rotates the spheres inside collide with the particles of the solid, thus crushing them towards smaller diameters. In the case of grinding, the solid particles are formed when the grinding units of the device rub against each other while particles of the solid are trapped in between. Methods like crushing and cutting can also be used for reducing particle diameter. Crushing employs hammer-like tools to break the solid into smaller particles by means of impact. Cutting uses sharp blades to cut the rough solid pieces into smaller ones. In addition, modern micronization methods that use supercritical fluids in the micronization process can be used. These methods use supercritical fluids to induce a state of supersaturation, which leads to precipitation of individual particles. Suitable techniques include the RESS process (Rapid Expansion of Supercritical Solutions), the SAS method (Supercritical Anti-Solvent) and the PGSS method (Particles from Gas Saturated Solutions). These modern techniques allow for greater tuneability of the process. Parameters like relative pressure and temperature, solute concentration, and antisolvent to solvent ratio can be varied to adjust to obtain the desired particle size. The supercritical fluid methods result in finer control over particle diameters, distribution of particle size and consistency of morphology.

In some embodiments, micronized 3,4-diaminopyridine or its salt suitable for use in the oral formulations of the present disclosure is a composition where 90% or more of the particles have a particle size of 20 microns or less (i.e., ≤20 μm). In some embodiments, the oral pharmaceutical formulations of the present disclosure comprise micronized 3,4-diaminopyridine phosphate salt. In some embodiments, 90% or more of the particles in the micronized 3,4-diaminopyridine phosphate salt have a particle size of 20 microns or less.

3,4-Diaminopyridine and its salts can be prepared by any suitable method known in the art. The free base is commercially available from, for example, Millipore Sigma (formerly Sigma-Aldrich). A tablet formulation of 3,4-diaminopyridine free base is sold by Jacobus Pharmaceutical Company, Inc. under the trademark Ruzurgi®. A tablet formulation of the phosphate salt is sold by Catalyst Pharmaceuticals, Inc. under the trademark Firdapse®.

### Dosage and Administration

In some aspects, the effective amount of 3,4-diaminopyridine or pharmaceutically acceptable salt thereof is administered to the subject at least once daily.

In some aspects, 3,4-diaminopyridine is administered as a free base. In some embodiments of this aspect, the effective amount of 3,4-diaminopyridine free base is about 5 mg to about 30 mg administered as a single dose. In some embodiments, the effective amount of 3,4-diaminopyridine free base is provided in a total daily dose ranging from about 15 mg to about 100 mg administered in one or more single doses per day. In some embodiments, the total daily dose of 3,4-diaminopyridine free base ranges from about 15 mg to about 80 mg administered in one or more single doses per day. In some embodiments, the total daily dose of 3,4-diaminopyridine fee base is administered in 1, 2, 3, 4, or 5 single doses per day. In some embodiment, the total daily dose of 3,4-diaminopyridine free base is administered in 3 or 4 single doses per day. In some embodiments, the single dose of 3,4-diaminopyridine free base is provided as one or more scored tablets or portions thereof. In some embodiments, the scored tablet comprises about 10 mg of 3,4-diaminopyridine free base.

In some aspects, 3,4-diaminopyridine is administered as a pharmaceutically acceptable salt.

In certain aspects, an effective amount of 3,4-diaminopyridine is administered as a phosphate salt at least once daily.

In some embodiments, the effective amount of 3,4-diaminopyridine phosphate salt is about 9.5 mg to about 56.9 mg administered as a single dose. In some embodiments, the effective amount is provided in a total daily dose ranging from about 28.5 mg to about 189.8 mg of 3,4-diaminopyridine phosphate salt administered in one or more single doses per day. In some embodiments, the total daily dose of 3,4-diaminopyridine phosphate salt ranges from about 28.5 mg to about 151.8 mg administered in one or more single doses per day. In some embodiment, the total daily dose of 3,4-diamonipyridine phosphate salt is administered in 1, 2, 3, 4, or 5 single doses per day. In some embodiments, the total daily dose of 3,4-diaminopyridine phosphate salt is administered in 3 or 4 single doses per day.

In certain embodiments, the single dose is provided as one or more scored tablets or portions thereof comprising 3,4-diaminopyridine phosphate salt. In some embodiments, the scored tablet comprises about 18.98 mg of 3,4-diaminopyridine phosphate salt.

### EXAMPLES

The method of treatment described herein is now further detailed with reference to the following examples. These examples are provided for the purpose of illustration only and the embodiments described herein should in no way be construed as being limited to these examples. Rather, the embodiments should be construed to encompass any and all variations which become evident as a result of the teaching provided herein.

### Example 1

A female subject self-reported her sexual functioning for the past 4 weeks using the FSFI questionnaire (“the pre-treatment FSFI”). According to the pre-treatment FSFI, 1) the subject sometimes (about half the time) felt sexual desire or interest and rated her level of sexual desire or interest low, 2) the subject felt sexually aroused a few times (less than half the time) and rated the level of sexual arousal low, 3) the subject had very low or no confidence about becoming sexually aroused during sexual activity or intercourse; 4) the subject was never or almost never satisfied with her arousal during sexual activity or intercourse, 5) the subject became almost never lubricated during sexual activity or intercourse and almost never or never maintained her lubrication until completion of sexual activity or intercourse, 6) it was very difficult for the subject to become lubricated and to maintain her lubrication until completion of sexual activity or intercourse, 7) the subject almost never or never reached orgasm when having sexual stimulation or intercourse, 8) it was extremely difficult or impossible for the subject to reach orgasm when having sexual activity or intercourse, 9) the subject was very dissatisfied with her ability to reach orgasm during sexual activity or intercourse, with the amount of emotional closeness during sexual activity between her and her partner, with her sexual relationship with her partner, and with her overall sexual life.

The subject then orally self-administered a total daily dose of 80 mg of 3,4-diaminopyridine phosphate salt for 4 weeks. The last dose of each day was self-administered at about 5:30 pm. The timing of her sexual activity, if any, was in the evening.

After the 4-week treatment period, the subject self-reported her sexual functioning for the past 4 weeks using the FSFI questionnaire (“the post-treatment FSFI”). According to the post-treatment FSFI, 1) the subject almost always or always felt sexual desire or interest and rated her level of sexual desire or interest moderate, 2) the subject felt sexually aroused almost always or always and rated the level of sexual arousal high, 3) the subject had very high confidence about becoming sexually aroused during sexual activity or intercourse; 4) the subject was most times (more than half the time) satisfied with her arousal during sexual activity or intercourse, 5) the subject became most times (more than half the time) lubricated during sexual activity or intercourse and also most times maintained her lubrication until completion of sexual activity or intercourse, 6) it was slightly difficult for the subject to become lubricated and to maintain her lubrication until completion of sexual activity or intercourse, 7) the subject most times reached orgasm when having sexual stimulation or intercourse, 8) it was not difficult for the subject to reach orgasm when having sexual activity or intercourse, 9) the subject was very satisfied with her ability to reach orgasm during sexual activity or intercourse, with the amount of emotional closeness during sexual activity between her and her partner, with her sexual relationship with her partner, and with her overall sexual life.

The subject felt better emotional experience with her partner and she was more confident while being treated as described above. The subject also reported that the time to reach orgasm was shorter after taking 3,4-diaminopyridine phosphate salt.

### Example 2

A female subject self-reported her sexual functioning for the past 4 weeks using the FSFI questionnaire (“the pre-treatment FSFI”). According to the pre-treatment FSFI, 1) the subject sometimes (about half the time) felt sexual desire or interest and rated her level of sexual desire or interest moderate, 2) the subject felt sexually aroused sometimes (about half the time) and rated the level of sexual arousal moderate, 3) the subject had moderate confidence about becoming sexually aroused during sexual activity or intercourse; 4) the subject was most times (more than half the time) satisfied with her arousal during sexual activity or intercourse, 5) the subject became sometimes lubricated during sexual activity or intercourse and sometimes maintained her lubrication until completion of sexual activity or intercourse, 6) it was slightly difficult for the subject to become lubricated and not difficult to maintain her lubrication until completion of sexual activity or intercourse, 7) the subject almost always or always reached orgasm when having sexual stimulation or intercourse, 8) it was not difficult or impossible for the subject to reach orgasm when having sexual activity or intercourse, 9) the subject was moderately satisfied with her ability to reach orgasm during sexual activity or intercourse, and with the amount of emotional closeness during sexual activity between her and her partner, and 10) the subject was about equally satisfied and dissatisfied with her sexual relationship with her partner, and with her overall sexual life.

The subject then orally self-administered a total daily dose of 60 mg of 3,4-diaminopyridine phosphate salt for 4 weeks. The last dose of each day was self-administered at about 6 pm. The timing of her sexual activity, if any, was in the evening at 8-9 pm.

After the 4-week treatment period, the subject self-reported her sexual functioning for the past 4 weeks using the FSFI questionnaire (“the post-treatment FSFI”). According to the post-treatment FSFI, 1) the subject almost always or always felt sexual desire or interest and rated her level of sexual desire or interest very high, 2) the subject felt sexually aroused most times (more than half the time) and rated the level of sexual arousal very high, 3) the subject had very high confidence about becoming sexually aroused during sexual activity or intercourse; 4) the subject was almost always or always satisfied with her arousal during sexual activity or intercourse, 5) the subject became almost always or always lubricated during sexual activity or intercourse and almost never or never maintained her lubrication until completion of sexual activity or intercourse, 6) it was not difficult for the subject to become lubricated and to maintain her lubrication until completion of sexual activity or intercourse, 7) the subject almost always or always reached orgasm when having sexual stimulation or intercourse, 8) it was not difficult for the subject to reach orgasm when having sexual activity or intercourse, 9) the subject was very satisfied with her ability to reach orgasm during sexual activity or intercourse, and 10) the subject was very satisfied with the amount of emotional closeness during sexual activity between her and her partner, with her sexual relationship with her partner, and with her overall sexual life.

The subject reported that she felt better emotional experience with her partner and she was more confident while being treated as described above. The subject also reported that the time to reach orgasm was shorter after taking 3,4-diaminopyridine phosphate salt.

### Example 3

A female subject self-reported her sexual functioning for the past 4 weeks using the FSFI questionnaire (“the pre-treatment FSFI”). According to the pre-treatment FSFI, 1) the subject sometimes (about half the time) felt sexual desire or interest and rated her level of sexual desire or interest moderate, 2) the subject felt sexually aroused most times (more than half the time) and rated the level of sexual arousal moderate, 3) the subject had high confidence about becoming sexually aroused during sexual activity or intercourse; 4) the subject was sometimes (about half the time) satisfied with her arousal during sexual activity or intercourse, 5) the subject became sometimes lubricated during sexual activity or intercourse and sometimes maintained her lubrication until completion of sexual activity or intercourse, 6) it was slightly difficult for the subject to become lubricated and slightly difficult to maintain her lubrication until completion of sexual activity or intercourse, 7) the subject almost always or always reached orgasm when having sexual stimulation or intercourse, 8) it was slightly difficult for the subject to reach orgasm when having sexual activity or intercourse, 9) the subject was moderately dissatisfied with her ability to reach orgasm during sexual activity or intercourse, and very satisfied with the amount of emotional closeness during sexual activity between her and her partner, and 10) the subject was moderately satisfied with her sexual relationship with her partner, and with her overall sexual life.

The subject then orally self-administered a total daily dose of 40 mg of 3,4-diaminopyridine phosphate salt for 4 weeks. The last dose of each day was self-administered at about 11 pm (5 mg). The timing of her sexual activity, if any, was in the morning, afternoon, and at night.

After the 4-week treatment period, the subject self-reported her sexual functioning for the past 4 weeks using the FSFI questionnaire (“the post-treatment FSFI”). According to the post-treatment FSFI, 1) the subject almost always or always felt sexual desire or interest and rated her level of sexual desire or interest very high, 2) the subject felt sexually aroused almost always or always and rated the level of sexual arousal very high, 3) the subject had very high confidence about becoming sexually aroused during sexual activity or intercourse; 4) the subject was almost always or always satisfied with her arousal during sexual activity or intercourse, 5) the subject became almost always or always lubricated during sexual activity or intercourse and almost always or always maintained her lubrication until completion of sexual activity or intercourse, 6) it was not difficult for the subject to become lubricated and to maintain her lubrication until completion of sexual activity or intercourse, 7) the subject almost always or always reached orgasm when having sexual stimulation or intercourse, 8) it was not difficult for the subject to reach orgasm when having sexual activity or intercourse, 9) the subject was very satisfied with her ability to reach orgasm during sexual activity or intercourse, and 10) the subject was very satisfied with the amount of emotional closeness during sexual activity between her and her partner, with her sexual relationship with her partner, and with her overall sexual life.

The subject reported that she experienced multiple orgasms after taking 3,4-diaminopyridine phosphate salt, which she had not experienced before taking 3,4-diaminopyridine phosphate salt.

### Example 4

A 70-year old male subject self-reported his sexual functioning for the past 4 weeks using the IIEF questionnaire (“the pre-treatment IIEF”). The pre-treatment IIEF for the subject revealed the following: 1) the subject was able to get an erection during sexual activity a few times (less than half the time) (score 2), 2) when the subject had erections with sexual stimulation, the erections were almost never or never hard enough for penetration (score 1), 3) the subject was able to penetrate (enter) his partner a few times (less than half the time) when he attempted intercourse (score 2); 4) during sexual intercourse, the subject was almost never or never able to maintain his erection after he had penetrated his partner (score 1), 5) during sexual intercourse, it was extremely difficult for the subject to maintain his erection to completion of intercourse (score 1), 6) the subject had three to four attempts for sexual intercourse (score 2), 7) when the subject attempted sexual intercourse, it was almost never or never satisfactory for him (score 1), 8) sexual intercourse was not very enjoyable for the subject (score 2), 9) when the subject had sexual stimulation or intercourse, he ejaculated a few times (less than half the time) (score 2), 10) when the subject had sexual stimulation or intercourse, he had the feeling of orgasm or climax a few times (less than half the time) (score 2); 11) the subject felt sexual desire sometimes (about half the time) (score 3); 12) the level of his sexual desire was low (score 2); 13) the subject has been moderately dissatisfied with his overall sex life (score 2); 14) the subject has been moderately dissatisfied with his sexual relationship with his partner (score 2); and 15) the subject's confidence that he could get and keep an erection was very low (score 1).

The subject then orally self-administered a total daily dose of 60 mg (4 times 15 mg) of 3,4-diaminopyridine phosphate salt for 4 weeks. The last dose of each day was self-administered at about 8:30 pm. The timing of his sexual activity, if any, was at about 11:30 am (after the 11 am dose) or at about 4 pm.

After the 4-week treatment period, the subject self-reported his sexual functioning for the past 4 weeks using the IIEF questionnaire (“the post-treatment IIEF”). According to the post-treatment IIEF, 1) the subject was able to get an erection during sexual activity most times (more than half the time) (score 4), 2) when the subject had erections with sexual stimulation, the erections were most times (more than half the time) hard enough for penetration (score 4), 3) the subject was able to penetrate (enter) his partner most times (more than half the time) when he attempted intercourse (score 4); 4) during sexual intercourse, the subject was sometimes (about half the time) able to maintain his erection after he had penetrated his partner (score 3), 5) during sexual intercourse, it was slightly difficult for the subject to maintain his erection to completion of intercourse (score 4), 6) the subject had five to six attempts for sexual intercourse (score 3), 7) when the subject attempted sexual intercourse, it was most times (more than half the time) satisfactory for him (score 4), 8) sexual intercourse was highly enjoyable for the subject (score 4), 9) when the subject had sexual stimulation or intercourse, he ejaculated most times (more than half the time) (score 4), 10) when the subject had sexual stimulation or intercourse, he had the feeling of orgasm or climax most times (more than half the time) (score 4); 11) the subject felt sexual desire almost always or always (score 5); 12) the level of his sexual desire was high (score 4); 13) the subject has been moderately satisfied with his overall sex life (score 4); 14) the subject has been moderately satisfied with his sexual relationship with his partner (score 4); and 15) the subject's confidence that he could get and keep an erection was high (score 4).

The subject also reported that his overall sexual performance was better and that he was more aroused after taking 3,4-diaminopyridine phosphate salt.

The subject's pre-treatment IIEF and post-treatment IIEF scores in the five functional domains (erectile function, orgasmic function, sexual desire, intercourse satisfaction and overall satisfaction) are provided below in Table 3. The results in Table 3 clearly show that treatment with 3,4-diaminopyridine phosphate salt improved the subject's all the 5 domains of male sexual function. The post-treatment scores for each functional domain are similar to those presented in Table 2 above for the control group of normal volunteers.

TABLE 3

| | | Pre- | Post- |
|---|---|---|---|
| | Maximum | Treatment | Treatment |
| Function Domain | Score | Score | Score |
| A. Erectile Function (questions 1-5 | 30 | 8 | 23 |
| and 15) | | | |
| B. Orgasmic Function (questions 9 | 10 | 4 | 8 |
| and 10) | | | |
| C. Sexual Desire (questions 11 and | 10 | 5 | 9 |
| 12) | | | |
| D. Intercourse Satisfaction (questions | 15 | 5 | 11 |
| 6-8) | | | |
| E. Overall Satisfaction (questions 13 | 10 | 4 | 8 |
| and 14) | | | |

### Example 5

A 65-year old male subject self-reported his sexual functioning for the past 4 weeks using the IIEF questionnaire (“the pre-treatment IIEF”). The pre-treatment IIEF for the subject revealed the following: 1) the subject was able to get an erection during sexual activity a few times (less than half the time) (score 2), 2) when the subject had erections with sexual stimulation, the erections were almost never or never hard enough for penetration (score 1), 3) the subject was able to penetrate (enter) his partner a few times (less than half the time) when he attempted intercourse (score 2); 4) during sexual intercourse, the subject was almost never or never able to maintain his erection after he had penetrated his partner (score 1), 5) during sexual intercourse, it was extremely difficult for the subject to maintain his erection to completion of intercourse (score 1), 6) the subject had three to four attempts for sexual intercourse (score 2), 7) when the subject attempted sexual intercourse, it was almost never or never satisfactory for him (score 1), 8) sexual intercourse was not very enjoyable for the subject (score 2), 9) when the subject had sexual stimulation or intercourse, he ejaculated a few times (less than half the time) (score 2), 10) when the subject had sexual stimulation or intercourse, he had the feeling of orgasm or climax a few times (less than half the time) (score 2); 11) the subject felt sexual desire sometimes (about half the time) (score 3); 12) his level of sexual desire was low (score 2); 13) the subject has been moderately dissatisfied with his overall sex life (score 2); 14) the subject has been moderately dissatisfied with his sexual relationship with his partner (score 2); and 15) the subject's confidence that he could get and keep an erection was very low (score 1).

The subject then orally self-administered a total daily dose of 80 mg (4 times 20 mg) of 3,4-diaminopyridine phosphate salt for 4 weeks. The last dose of each day was self-administered at about 6:30 pm. The timing of his sexual activity, if any, was at about 7:30 pm.

After the 4-week treatment period, the subject self-reported his sexual functioning for the past 4 weeks using the IIEF questionnaire (“the post-treatment IIEF”). According to the post-treatment IIEF, 1) the subject was able to get an erection during sexual activity most times (more than half the time) (score 4), 2) when the subject had erections with sexual stimulation, the erections were most times (more than half the time) hard enough for penetration (score 4), 3) the subject was able to penetrate (enter) his partner most times (more than half the time) when he attempted intercourse (score 4); 4) during sexual intercourse, the subject was sometimes (about half the time) able to maintain his erection after he had penetrated his partner (score 3), 5) during sexual intercourse, it was slightly difficult for the subject to maintain his erection to completion of intercourse (score 4), 6) the subject had five to six attempts for sexual intercourse (score 3), 7) when the subject attempted sexual intercourse, it was most times (more than half the time) satisfactory for him (score 4), 8) sexual intercourse was highly enjoyable for the subject (score 4), 9) when the subject had sexual stimulation or intercourse, he ejaculated most times (more than half the time) (score 4), 10) when the subject had sexual stimulation or intercourse, he had the feeling of orgasm or climax most times (more than half the time) (score 4); 11) the subject felt sexual desire almost always or always (score 5); 12) his level of sexual desire was high (score 4); 13) the subject has been moderately satisfied with his overall sex life (score 4); 14) the subject has been moderately satisfied with his sexual relationship with his partner (score 4); and 15) the subject's confidence that he could get and keep an erection was high (score 4).

The subject also reported that his overall sexual performance was better after taking 3,4-diaminopyridine phosphate salt.

The subject's pre-treatment IIEF and post-treatment IIEF scores in the five functional domains (erectile function, orgasmic function, sexual desire, intercourse satisfaction and overall satisfaction) were identical to those provided in Table 3 for the 70-year old male subject.

Having now fully described this disclosure, it will be understood by those of ordinary skill in the art that the same can be performed within a wide and equivalent range of conditions, formulations, and other parameters without affecting the scope of the invention or any embodiment thereof.

Other embodiments of the disclosure will be apparent to those skilled in the art from consideration of the specification and practice of the invention disclosed herein. It is intended that the specification be considered exemplary only, with a true scope and spirit of the invention being indicated by the following claims.

All patents, patent applications, and publications cited herein are fully incorporated by reference herein in their entirety.

What is claimed is:

1. A method of treating sexual dysfunction in a subject in need thereof, comprising orally administering to the subject an effective amount of 3,4-diaminopyridine phosphate salt.

2. The method of claim 1, wherein the sexual dysfunction is a disorder selected from the group consisting of female sexual interest/arousal disorder, female orgasmic disorder, other female sexual dysfunction, male hypoactive sexual desire disorder, delayed ejaculation, erectile disorder, and other male sexual dysfunction.

3. The method of claim 1 or 2, wherein the subject is a female.

4. The method of claim 2, wherein the female orgasmic disorder is anorgasmia.

5. The method of claim 4, wherein the anorgasmia is selected from the group consisting of situational anorgasmia, primary anorgasmia, and secondary anorgasmia.

6. The method of claim 1 or 2, wherein the subject is a male.

7. The method of claim 6, wherein the male sexual dysfunction is a primary sexual dysfunction.

8. The method of claim 6, wherein the male sexual dysfunction is a secondary sexual dysfunction.

9. The method of claim 8, wherein the secondary sexual dysfunction is erectile disorder.

10. The method of claim 8 or 9, wherein the secondary sexual dysfunction is caused by an autonomic dysfunction, a cardiac-related condition, or a blood-flow affecting medication.

11. The method of claim 10, wherein the sexual dysfunction is caused by an autonomic dysfunction.

12. The method of claim 11, wherein the autonomic dysfunction is caused by diabetes, multiple sclerosis, Parkinson's disease, brain or spinal tumor, stroke, temporal lobe epilepsy, an autoimmune disease, or alcoholism.

13. The method of claim 12, wherein the autonomic dysfunction is diabetic autonomic neuropathy.

14. A method of increasing sexual desire in a subject in need thereof, comprising orally administering to the subject an effective amount of 3,4-diaminopyridine phosphate salt.

15. A method of increasing orgasmic ability in a subject in need thereof, comprising orally administering to the subject an effective amount of 3,4-diaminopyridine phosphate salt.

16. A method of increasing sexual satisfaction in a subject in need thereof, comprising orally administering to the subject an effective amount of 3,4-diaminopyridine phosphate salt.

17. The method of any one of claims 14-16, wherein the subject is a female subject.

18. The method of claim 3 or 17, wherein the female subject is a pre-menopausal, perimenopausal, menopausal or post-menopausal woman.

19. The method of any one of claims 14-16, wherein the subject is a male subject.

20. The method of any one of claims 1-19, wherein the effective amount of 3,4-diaminopyridine phosphate salt is administered in a pharmaceutical formulation suitable for oral administration.

21. The method of claim 20, wherein the pharmaceutical formulation is a solid, a semi-solid or a liquid oral formulation.

22. The method of claim 21, the solid oral formulation is selected from the group consisting of a tablet, a pill, a dragee, a powder, and capsule.

23. The method of any one of claims 1-22, wherein the effective amount of 3,4-diaminopyridine phosphate salt is administered at least once daily.

24. The method of any one of claims 1-23, wherein the effective amount of 3,4-diaminopyridine phosphate salt is about 9.5 mg to about 56.9 mg administered as a single dose.

25. The method of claim 24, wherein the effective amount is provided in a total daily dose ranging from about 28.5 mg to about 189.8 mg of 3,4-diaminopyridine phosphate salt administered in one or more single doses per day.

26. The method of claim 25, wherein the total daily dose ranges from about 28.5 mg to about

151. 8 mg of 3,4-diaminopyridine in one or more single doses per day.

27. The method of claim 25 or 26, wherein the total daily dose is administered in 1, 2, 3, 4, or 5 single doses per day.

28. The method of claim 27, wherein the total daily dose is administered in 3 or 4 single doses per day.

29. The method of any one of claims 24-28, wherein the single dose is provided as one or more scored tablets or portions thereof comprising 3,4-diaminopyridine phosphate salt.

30. The method of claim 29, wherein the scored tablet comprises about 18.98 mg of 3,4-diaminopyridine phosphate salt.
